(19) United States
(12) Patent Application Publication
(10) Pub. No.: US 2022/0202469 A1
(43) Pub. Date: Jun. 30, 2022

(54) Pre-Treatment Protocol Using Topical Anesthetic and Cooling

(71) Applicant: Accure Acne, Inc., Boulder, CO (US)

(72) Inventors: Aubrey Jean Eck, Denver, CO (US); Michael John Estes, Lafayette, CO (US); Henrik Hofvander, Budapest (HU)

(73) Assignee: Accure Acne, Inc., Boulder, CO (US)

(21) Appl. No.: 17/564,836

(22) Filed: Dec. 29, 2021

Related U.S. Application Data
(60) Provisional application No. 63/131,781, filed on Dec. 29, 2020.

(51) Int. Cl. A61B 18/02; A61B 18/20; A61K 9/00; A61K 31/167; A61K 31/137; A61B 90/00

(52) U.S. Cl. CPC A61B 18/0218; A61B 18/203; A61K 9/0014; A61B 2018/00452; A61K 31/137; A61B 90/04; A61K 31/167

(57) ABSTRACT

The present disclosure is directed to a method of performing a photo-thermal dermatological procedure without use of an injectable anesthetic. The method includes applying a topical anesthetic to a treatment area of skin, measuring a surface temperature of the treatment area of skin, and passing a stream of cold air over the area of skin such that the surface temperature of the treatment area of skin is maintained between 0 degrees Celsius and 15 degrees Celsius for a duration of at least 30 seconds. The method further includes administering a photo-thermal energy dose to the area of skin after the duration.

### FIELD OF THE INVENTION

The present invention relates to energy-based treatments and, more specifically, systems and methods for preparing a treatment area prior to treatment without the use of an injectable anesthetic.

### BACKGROUND OF THE INVENTION

Sebaceous glands and other chromophores embedded in a medium such as the dermis, can be treated using thermal damage by heating the chromophore with a targeted light source, such as a laser. However, the application of enough thermal energy to damage the chromophore can also result in undesirable damage to the surrounding dermis and the overlying epidermis, thus leading to epidermis and dermis damage, as well as possible pain to the patient during treatment.

A commonly used technique to alleviate pain during such energy-based treatments is to inject a local anesthetic at and around the treatment area. However, particularly for the treatment of sensitive locations such as the face, the process of repeatedly injecting the local anesthetic over the treatment area can itself be more painful than the energy-based treatment itself.

### SUMMARY OF THE DISCLOSURE

The following presents a simplified summary of one or more aspects in order to provide a basic understanding of such aspects. This summary is not an extensive overview of all contemplated aspects, and is intended to neither identify key or critical elements of all aspects nor delineate the scope of any or all aspects. Its purpose is to present some concepts of one or more aspects in a simplified form as a prelude to the more detailed description that is presented later.

In an aspect, the present disclosure is directed to a method of performing a photo-thermal dermatological procedure without use of an injectable anesthetic. The method includes applying a topical anesthetic to a treatment area of skin, and then passing a stream of cold air over the area of skin such that the surface temperature of the treatment area of skin is maintained between 0 degrees Celsius and 15 degrees Celsius for a duration of at least 30 seconds. The surface temperature of the skin is either directly measured during the cooling, or the temperature profile is known due to experimental testing. The method further includes administering a photo-thermal energy dose to the area of skin after the duration.

In another aspect, the present disclosure is directed to a method of performing a dermatological procedure without use of an injectable anesthetic. The method includes applying a topical anesthetic to a treatment area of skin, and cooling the area of skin such that the surface temperature of the treatment area of skin is maintained between 0 degrees Celsius and 15 degrees Celsius for a duration of at least 20 seconds. The surface temperature of the skin is either directly measured during the cooling, or the temperature profile is known due to experimental testing. The method further includes performing a dermatological procedure on the area of skin after the duration.

### BRIEF DESCRIPTION OF THE DRAWINGS

The appended drawings illustrate only some implementation and are therefore not to be considered limiting of scope.

FIG. 1 illustrates an exemplary photothermal targeting treatment system, in accordance with an embodiment.

FIG. 2 illustrates an exemplary scanner arrangement for use with the photothermal targeting treatment system, in accordance with an embodiment.

FIG. 3 illustrates an exemplary data flow scheme between various components involved in carrying out a dermatological pre-treatment protocol, in accordance with an embodiment.

FIG. 4 illustrates method steps associated with a dermatological pre-treatment protocol, in accordance with an embodiment.

FIG. 5 shows a flow chart illustrating an exemplary process for analyzing a measured skin surface temperature, predicting a temperature of the skin when subsequent laser pulses and/or additional cooling are applied, and modifying the treatment protocol, in accordance with an embodiment.

FIG. 6 shows pain score results associated with performing a dermatological treatment after performing different pre-treatment protocols.

### DETAILED DESCRIPTION

The present invention is described more fully hereinafter with reference to the accompanying drawings, in which embodiments of the invention are shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. In the drawings, the size and relative sizes of layers and regions may be exaggerated for clarity.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another region, layer or section. Thus, a first element, component, region, layer or section discussed below could be termed a second element, component, region, layer or section without departing from the teachings of the present invention.

Spatially relative terms, such as “beneath,” “below,” “lower,” “under,” “above,” “upper,” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” or “under” other elements or features. Thus, the exemplary terms “below” and “under” can encompass both an orientation of above and below. The device may be otherwise oriented (rotated 90 degrees or at other orientations) and the spatially relative descriptors used herein interpreted accordingly. In addition, it will also be understood that when a layer is referred to as “between” two layers, it can be the only layer between the two layers, or one or more intervening layers may also be present.

The terminology used herein is for the purpose of describing particular embodiments only and is not intended to be limiting of the invention. As used herein, the singular forms “a,” “an,” and “the” are intended to include the plural forms as well, unless the context clearly indicates otherwise. It will be further understood that the terms “comprises” and/or “compromising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items, and may be abbreviated as “/”.

It will be understood that when an element or layer is referred to as being “on,” “connected to,” “coupled to,” or “adjacent to” another element or layer, it can be directly on, connected, coupled, or adjacent to the other element or layer, or intervening elements or layers may be present. In contrast, when an element is referred to as being “directly on,” “directly connected to,” “directly coupled to,” or “immediately adjacent to” another element or layer, there are no intervening elements or layers present. Likewise, when light is received or provided “from” one element, it can be received or provided directly from that element or from an intervening element. On the other hand, when light is received or provided “directly from” one element, there are no intervening elements present.

Embodiments of the invention are described herein with reference to cross-section illustrations that are schematic illustrations of idealized embodiments (and intermediate structures) of the invention. As such, variations from the shapes of the illustrations as a result, for example, of manufacturing techniques and/or tolerances, are to be expected. Thus, embodiments of the invention should not be construed as limited to the particular shapes of regions illustrated herein but are to include deviations in shapes that result, for example, from manufacturing. Accordingly, the regions illustrated in the figures are schematic in nature and their shapes are not intended to illustrate the actual shape of a region of a device and are not intended to limit the scope of the invention.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

Many different types of dermatological procedures have been developed to achieve therapeutic and/or cosmetic results for patients. Common examples of such procedures include skin resurfacing, sebaceous gland ablation, hair removal using a laser or electrolysis, tattooing, tattoo removal, and varicose vein removal. Many such procedures involve at least some pain to the patient before and during the treatment. In some instances, the pain can be severe, especially when the treatment area is over part of the body where the skin is particularly sensitive.

To make treatments more tolerable to patients, various approaches have been used to reduce pain. For example, topical anesthetics—compounds typically containing lidocaine which may be mixed with epinephrine and/or prilocaine—can be applied to the surface of the skin (i.e., the epidermis) prior to performing dermatological procedures. Currently in the United States, topical anesthetics containing greater than 5% lidocaine require a prescription from a physician to obtain while creams containing less than 5% lidocaine are available on an over-the-counter basis without a prescription. Application of topical anesthetics can reduce pain, but topical anesthetics alone, whether prescription or over-the-counter, are often insufficient to make dermatological procedures tolerable to patients.

Likewise surface cooling via, for example, a cold-air device can make certain procedures more tolerable. However, for laser-based procedures, pre-cooling the skin alone may be insufficient to reduce pain to acceptable levels.

More effective pain relief can be achieved by using injectable anesthetics which are delivered to deeper layers of skin (e.g., the dermis). However, downsides to using injectable anesthetics are that a physician's prescription for the medication is typically required and, in some cases, a physician must directly administer or otherwise supervise the injection of the prescription anesthetic medication. Moreover, the injection itself causes pain to the patient and may require application of a topical anesthetic prior to the injection, thereby increasing complexity and cost of the pre-treatment protocol.

In clinical research performed by the inventors, various pain-relieving pre-treatment protocols were tested on patients undergoing sebaceous gland ablation for the treatment of acne. A 1726 nm laser was used to carry out the ablation procedure. Numerous different compounds of topical anesthetics were tested, as well as various ways of applying the topical anesthetics. Results of the research showed that the tested topical anesthetics alone were not effective at reducing laser-induced pain sufficiently for the ablation treatment to be tolerable. Results further showed that tested prescription topical anesthetic creams containing up to 30% lidocaine were not much more effective than over-the-counter topical anesthetic creams containing only 5% lidocaine. Tolerable pain levels for patients were achieved only by using prescription injectable anesthetics prior to the ablation treatment.

The inventors also investigated skin surface cooling. Numerous experiments testing different levels of cooling and different application times were performed. Results of the research showed that the tested surface cooling procedures alone were not effective at reducing laser-induced pain sufficiently for the ablation treatment to be tolerable.

In order to facilitate improved patient experience and to make dermatological treatments more accessible (e.g., less costly and more widely available) to patients, the inventors identified a need for an effective pain mitigation technique that does not require prescription medication or direct involvement of a physician. Thus, the inventors identified a need for an effective pain reducing pre-treatment protocol that does not require injectable anesthetics or prescription topical anesthetics and that can be performed with minimal additional clinical equipment.

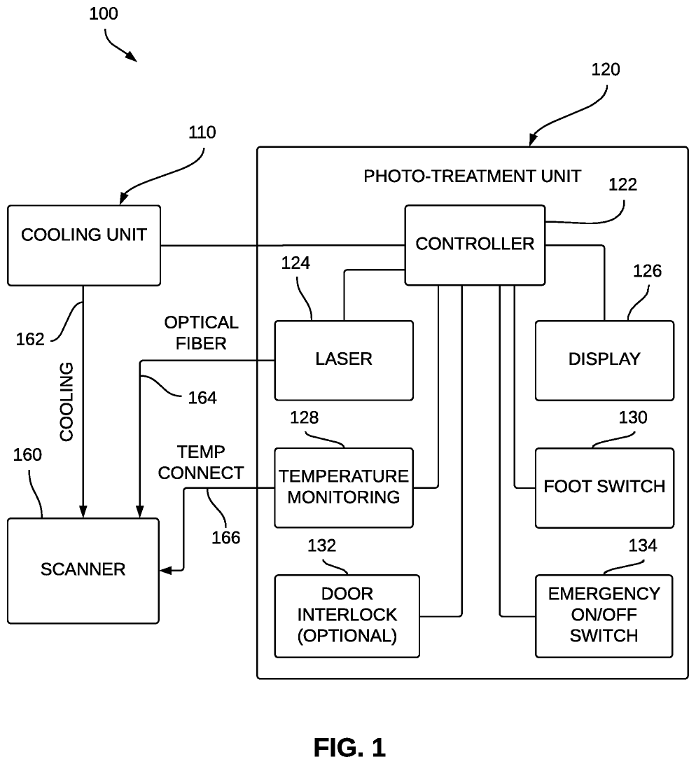

Referring initially to FIG. 1, an example a photo-thermal treatment system 100 is shown. Such a system may be used to complete one or more of the dermatological procedures previously discussed. For example, the system 100 can be used for photo-thermal ablation of sebaceous glands in a targeted fashion while sparing the epidermis and dermis layers of skin that overlay and surround the target sebaceous glands. The system 100 may be used to apply photo-thermal energy to a target, where the target may be one or more specific chromophores embedded in the skin. The target may be heated to a sufficiently high temperature by a photo-thermal energy source within the treatment system 100 so as to damage the target without damaging the surrounding medium.

Still referring to FIG. 1, the photo-thermal targeted treatment system 100 includes a cooling unit 110 and a photo-treatment unit 120. Cooling unit 110 provides a cooling mechanism for a cooling effect, such as by contact or by direct air cooling, to a treatment area, namely the outer skin layer area overlying a target sebaceous gland. Cooling unit 110 is coupled with a controller 122 within photo-treatment unit 120. It is noted that it is possible for the controller to be located within cooling unit 110 or to be located separately outside of both cooling unit 110 and photo treatment unit 122. As will be discussed herein below in further detail, the cooling unit 110 may be used in a pain reducing pre-treatment method prior to performing the photo-thermal targeted treatment.

Controller 122 further controls one or more other components that may be included within photo-treatment unit 120, such as a laser 124, a display 126, a temperature monitoring unit 128, a foot switch 130, an optional door interlock 132, and an emergency on/off switch 134. Laser 124 provides laser-based photo-thermal energy for the photo-treatment protocol, and controller 122 regulates the specific settings for the laser, such as the output power and pulse time settings. Laser 124 can be a single laser or a combination of two or more lasers. If more than one laser is used, the laser outputs are combined optically to function as one more powerful laser with photo-thermal energy from both lasers directed at the same target. Display 126 can include information such as the operating conditions of cooling unit 110, laser 124, and other system status information. Temperature monitoring unit 128 is used to measure and monitor the temperature of the skin surface in the treatment area, for example. Such temperature measurements may also be provided to a system user via the display 126.

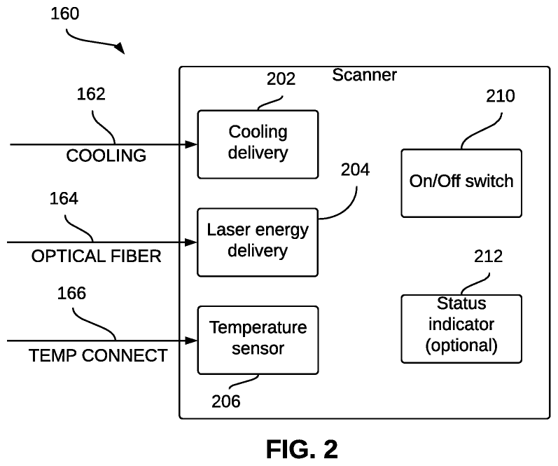

Referring to FIGS. 1 and 2 together, scanner 160 is shown. Scanner 160 is a device that can be positioned by a user with respect to a treatment area of a patient's skin. The scanner 160 may be hand-held and the user may be a physician, clinician, technician, or other trained individual. The scanner 160 is coupled with one or more modules or components that are included in the photo-treatment unit 120. For example, photo-thermal energy from the laser(s) 124 may travel to a laser energy delivery system 204 within the scanner 160 via an optical fiber 164. Temperature measurement data may be collected by a temperature sensor 206 on board the scanner 160 and transmitted to the temperature monitoring module 128 over a temperature connect line 166. Scanner 160 is also coupled with cooling unit 110 via a cooling line 162. In some embodiments, cooling unit 110 may deliver cold air to a cooling delivery system 202 within the scanner 160 through the cooling line 162. The scanner 160 may include an on/off switch 210, such as a trigger or button, which can receive input from a user and can be used to initiate a laser pulse delivery to the target area or provide instructions to other components within the system. The scanner 160 may further include an optional status indicator 212 which can indicate to a user on/off/ready/standby and/or other status modes as needed. For example, the status indicator 212 may be a light configured to change colors and/or blink to indicate a particular mode.

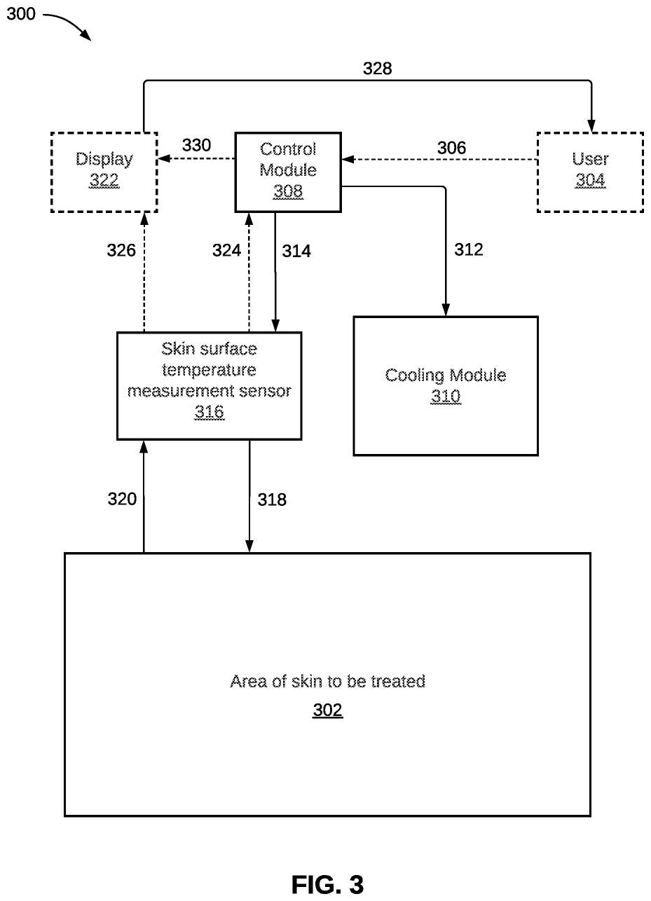

FIG. 3 illustrates an example flow of information through a system 300 that includes various components involved with a dermatological treatment. A treatment area of skin 302 is identified by a user 304. The user 304 may optionally provide input 306 (as represented by a dashed line) to a control module 308. The input 306 may represent instructions to turn on, turn off, or adjust a temperature or flow rate associated with cold air exiting a cooling module 310 via data connection 312 between the control module 308 and the cooling module 310. The input 306 may additionally or alternatively include instructions for the control module 308 to initiate a skin surface temperature measurement via a data connection 314 between the control module 308 and the skin surface temperature measurement sensor 316. The skin surface temperature measurement sensor 316 may receive instructions to take a measurement from the control module and may send a signal 318, such as an infrared light signal, toward the treatment area of skin 302. A signal 320 reflected from the skin 302 may be collected by the temperature measurement sensor 316 and may be interpreted as a skin surface temperature. The data may be relayed to the control module 308 and, optionally, a display component 322, via data connections 324, 326, respectively. The display 322 may indicate the measured surface temperature to the user 304 via an output signal 328, which may be a visual or audio output signal. The display 322 may additionally or alternatively display prompts or acknowledgements of instruction as provided by the control module 308 through data connection 330. One of skill in the art will appreciate that the various data connections described with respect to components within the system 300 may include wired and/or wireless connections.

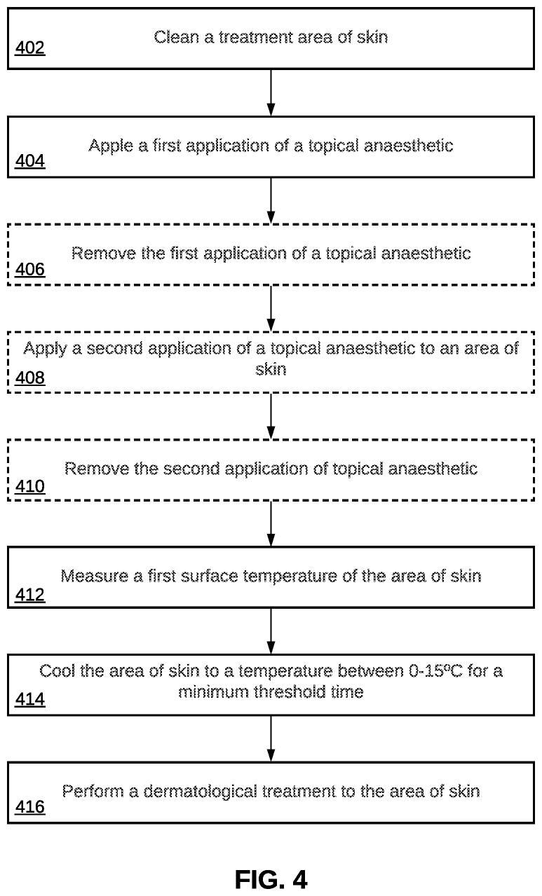

FIG. 4 illustrates steps of a method 400 that may be used prior to a dermatological procedure to reduce pain to the patient during the procedure. The method 400 may utilize a cooling module included within a dermatological treatment system similar to cooling modules 110, 310 as discussed with respect to FIGS. 1-3; however, alternative means for cooling the skin may be used instead of or in addition to such cooling modules as will be discussed herein below.

In a first step 402, a treatment area of skin is cleaned using, for example, an antiseptic to remove contaminants from the surface of the skin. In step 404, a topical anesthetic is applied to the area of skin. The topical anesthetic may be an anesthetic that can be obtained over-the-counter without requiring a physician's prescription. For example, the topical anesthetic may be a cream including up to approximately 5% lidocaine. The cream may further contain epinephrine and/or prilocaine. While topical anesthetics containing higher than 5% lidocaine may also be used, the inventors have achieved an unexpected result in that over-the-counter creams having up to approximately 5% lidocaine provide approximately the same amount of pain relief as prescription creams containing up to 30% lidocaine when used as part of the pre-treatment method 400 prior to performing a dermatological procedure.

In optional steps 406 and 408, indicated as optional by dashed lines, the first application of topical anesthetic may be removed and/or a second application of a topical anesthetic may be applied to the skin. The topical anesthetic applied in the second application may be the same or a different topical anesthetic compared to that used in the first application at step 404. The second application of topical anesthetic may optionally be removed at step 410. Additional optional anesthetic applications and removals may be performed on the area of skin as needed. In some embodiments, each application of topical anesthetic may include allowing the topical anesthetic to dwell on the skin for approximately 30 minutes prior to removal or prior to moving on to a next step of the pre-treatment method. In some embodiments, the total amount of dwell time over a plurality of topical anesthetic applications is at least 30 minutes. While the dwell time may include allowing the topical anesthetic to reside on the skin in open air, occlusion techniques may also be used. For example, the skin and topical anesthetic thereon may be covered by a plastic film, aluminum foil, or other similar materials for at least a portion of the dwell time. Additionally, the topical anesthetic may be massaged into the skin during at least a portion of the dwell time.

Following the sequence of one or more topical anesthetic applications and/or one or more removals of the topical anesthetic, a first skin surface temperature measurement of skin in the treatment area is taken at step 412. The first surface temperature measurement may be time stamped so as to track multiple subsequent skin surface temperature measurements over a period of time. Additionally or alternatively, the treatment area of skin may be correlated with a known skin temperature profile at step 412b. The known skin temperature profile may be used to estimate skin surface temperature based on one or more pre-cooling characteristics, such as air stream temperature, air stream flow rate, pre-cooling duration, ambient temperature, type of skin to be treated, or other characteristics. Using such a skin temperature profile may eliminate the need to perform multiple skin surface temperature measurements throughout the pre-cooling process.

Once the first surface temperature measurement is recorded and/or a skin temperature profile is correlated to the patient, the treatment area of skin is subjected to a cooling process. In some embodiments, the cooling process may include passing a stream of cold air over the area of skin such that the surface temperature of the area of skin is maintained within a range from approximately 0° C. to approximately 15° C. for a duration of at least 20 seconds at step 414. In some embodiments, the skin surface temperature may be maintained within the temperature range for at least 30 seconds. The lower bound of approximately 0° C. is selected in order to prevent tissue damage known to be caused when skin temperatures drop below 0° C. Other techniques for cooling the skin may be used in addition to or as an alternative to a cold air stream. For example, the patient's skin may be contacted by a cold surface or cold medium to conduct heat away and/or an evaporative medium may be applied one or more times to the patient's skin. Other known cooling techniques may also be used to cause skin surface temperature to stay within the 0-15° C. range for at least 30 seconds.

In some embodiments, multiple skin surface temperature measurements are obtained during the cooling process to confirm that the skin surface temperature is maintained within the 0° C. to 15° C. for a minimum selected time period. In some embodiments, skin surface temperature measurements may also be used as feedback to adjust the cooling process. For example, if the skin is approaching the upper or lower bounds of the desired temperature range, the rate of cooling by the cooling process may be increased or decreased, respectively. Such a feedback loop may utilize a control module, such as control module 308 as discussed with respect to FIG. 3, that can automatically make adjustments or indicate skin surface temperature levels to a user via a display so that the user can manually adjust the operation of the cooling module. In other embodiments, the skin temperature over time is known well enough, and is repeatable enough, given experimental results and known skin temperature profiles, that real-time temperature measurements are not required.

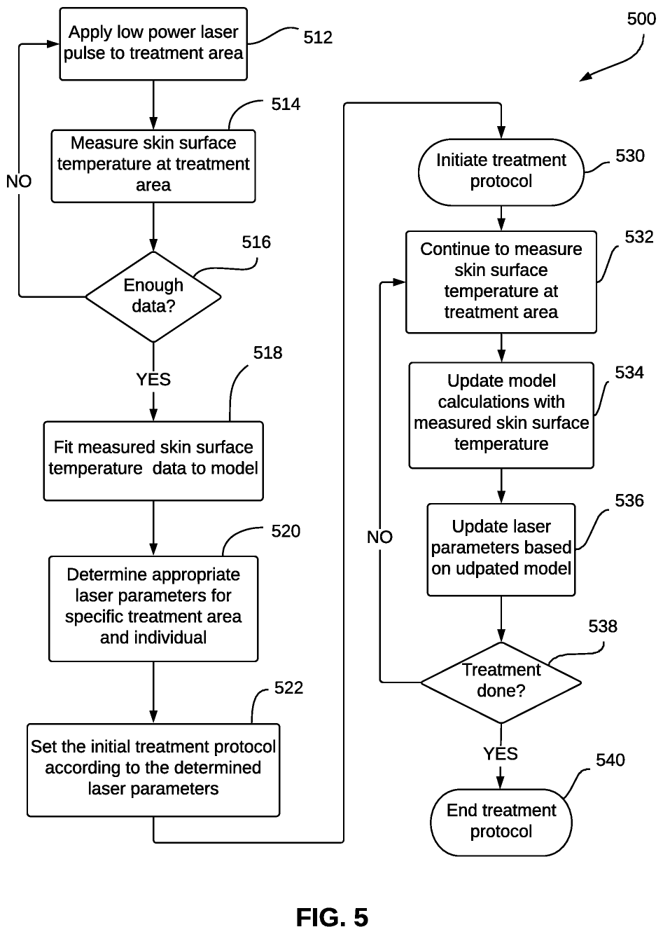

Once the skin has been cooled to a selected skin surface temperature for a desired period of time, the dermatological treatment may be performed with significantly reduced pain to the patient at step 416. In embodiments where the dermatological procedure includes laser ablation of sebaceous glands, the protocol 500 described in FIG. 5 may be followed. An analysis protocol 500 begins by applying a low power laser pulse to a treatment area of skin in a step 512. The laser power is set at values that are below the damage threshold for the epidermis. The skin surface temperature at the treatment area is then measured in a step 514. The temperature measurement can be performed, for example, using a low speed infrared camera or similar apparatus. A determination is made in a decision 516 whether enough data has been collected to fit the collected data into the pre-established correlation model that relates the laser power to skin surface temperature. If the answer to decision 516 is no, then the process returns to step 512, at which point a laser pulse at a different, low power setting is applied to the treatment area to gather additional correlation data between applied laser power and epidermis temperature.

If the answer to decision 516 is yes, then analysis protocol 500 proceeds to fit the measured skin surface temperature data to the established correlation model in a step 518. The appropriate laser parameters for the specific treatment area for the particular individual are determined in a step 520. Finally, in a step 522, the exact treatment protocol to be used for the specific treatment area for the particular individual is modified according to the appropriate laser parameters found in step 520.

Continuing to refer to FIG. 5, optionally, analysis protocol 500 can be continued during the actual treatment protocol. In an exemplary embodiment, following the setting of the laser parameters in step 522, a treatment protocol with the appropriate laser parameters is initiated in a step 530. Then, in a step 532, the process continues to measure the skin surface temperature at the treatment area. The measured skin surface temperatures are used to update the correlation model calculations in a step 534, and the laser parameters for the treatment protocol are updated based on the updated calculations in a step 536. Then a decision 538 is made to determine whether the treatment protocol (i.e., the number of laser pulses to be applied to the treatment area) is complete. If the answer to decision 538 is NO, then the analysis protocol returns to step 532 to continue measuring the skin surface temperature. If the answer to decision 538 is YES, then the treatment protocol is terminated in a step 540.

In other words, until the treatment protocol is complete, analysis protocol 500 can implement optional steps 530 through 540 to continue adjusting the laser parameters even during the actual treatment protocol. In fact, if other relevant data regarding the subject, such as laser settings from prior treatments in the same treatment area for the same subject, exist, they can also be fed into the model calculations for further refinement of the laser parameters.

Using the pre-treatment protocol described with respect to FIG. 4 prior to completing an energy-based dermatological procedure, such as sebaceous gland ablation, microwave treatments, photo-thermal treatments, and electro-needling yields significantly and unexpectedly reduced pain scores compared with known pre-treatment methods. Other types of energy-based dermatological procedures include laser-based dermatological treatments, such as those described in “Lasers in Dermatology” (https://www.dermnetnz.org/topics/lasers-in-dermatology/accessed 2020 Dec. 29).

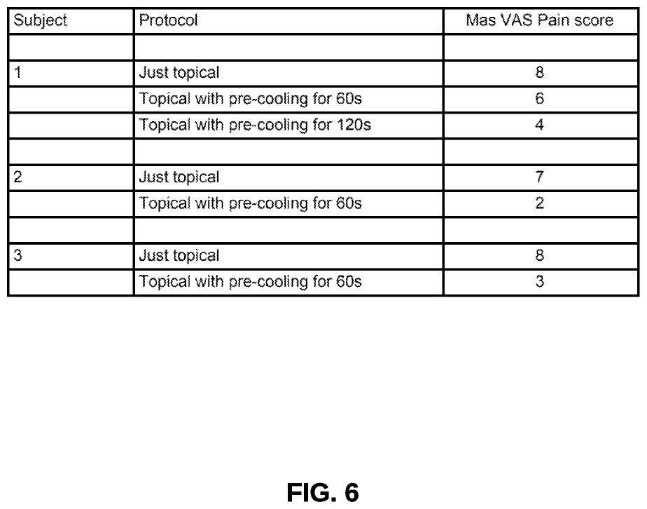

Referring to FIG. 6, chart 600 shows pain scores associated with various pre-treatment protocols for three subjects. Pain was measured using a Visual Analog Scale (VAS) pain rating method, which characterized pain over a scale from zero to ten, with zero representing no pain and ten representing extreme pain.

Subject 1 experienced four pre-treatment protocols before four rounds of laser-based sebaceous gland ablation. The first two protocols included the application of a topical anesthetic only, the topical anesthetic containing 5% lidocaine, and the subsequent laser-based treatments resulted in an average pain score of 7.0. The second two protocols included the 5% lidocaine topical anesthetic followed by pre-cooling for 120 seconds and resulted in an average pain score of 2.8.

Similarly, Subject 2 experienced pre-treatment protocols prior to laser-based sebaceous gland ablation. The first protocol included application of pre-cooling only. Subject 2 reported an associated pain score of over 7. The second protocol included application of 5% lidocaine topical anesthetic followed by pre-cooling for 150 seconds. Under this pre-treatment protocol, Subject 2 reported a pain score of 0.9.

In view of the experimental results reported with respect to FIG. 6, the pre-treatment protocol including application of a non-prescription topical anesthetic in combination with skin cooling significantly reduced pain scores for the subjects. While the amount of pain experienced by each subject may vary due to various factors including overall pain tolerance and area of skin on which the dermatological procedure is performed, the amount of pain each subject reported after receiving a topical anesthetic in combination with pre-cooling was significantly reduced with respect to a pre-treatment protocol that relied on topical anesthetics alone or a pre-treatment protocol that relied on cooling alone. Thus, the pre-treatment method described with respect to FIG. 4 is an effective way to reduce pain associated with a dermatological procedure without requiring the use of prescription medications, such as injectable anesthetics or prescription strength topical anesthetics, and may not require additional clinical equipment. Thus, the described pre-treatment method can significantly reduce patient pain, thereby improving tolerance of dermatological procedures by patients without the burden of expensive medications and equipment. Furthermore, since prescription medications are not required, the pre-treatment protocol may be completed by trained individuals with or without a medical license.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention.

Accordingly, many different embodiments stem from the above description and the drawings. It will be understood that it would be unduly repetitious and obfuscating to literally describe and illustrate every combination and subcombination of these embodiments. As such, the present specification, including the drawings, shall be construed to constitute a complete written description of all combinations and subcombinations of the embodiments described herein, and of the manner and process of making and using them, and shall support claims to any such combination or subcombination.

What is claimed is:

1. A method for performing an energy-based dermatological procedure without use of an injectable anesthetic, the method comprising:
applying a topical anesthetic to an area of skin;
passing a stream of cold air over the area of skin such that a surface temperature of the area of skin is maintained between 0 degrees Celsius and 15 degrees Celsius for a duration of at least 30 seconds; and
administering an energy dose to the area of skin after the duration.

2. The method of claim 1, wherein applying the topical anesthetic to the area of skin comprises applying a first application of topical anesthetic and applying at least a second application of topical anesthetic.

3. The method of claim 2, further comprising cleaning the area of skin between applying the first application of topical anesthetic and applying the second application of topical anesthetic.

4. The method of claim 1, wherein the surface temperature of the skin is measured in real-time.

5. The method of claim 1, further comprising massaging the topical anesthetic into the area of skin for at least 20 seconds.

6. The method of claim 1, wherein applying the topical anesthetic comprises allowing the topical anesthetic to dwell on the area of skin for at least 30 minutes.

7. The method of claim 1, further comprising occluding the topical anesthetic on the area of skin.

8. The method of claim 1, further comprising removing the topical anesthetic from the area of skin prior to cooling the area of skin.

9. The method of claim 1, wherein the skin is between 0 degrees and 15 degrees Celsius for at least 30 seconds.

10. The method of claim 1, wherein an active ingredient of the topical anesthetic comprises at least one selected from a group consisting of lidocaine, epinephrine, and prilocaine.

11. The method of claim 1, wherein administering the photo-thermal energy dose comprises administering at least a first laser pulse to the area of skin.

12. The method of claim 1, further comprising displaying a measured surface temperature of the area of skin and a total time that the skin surface has been below a targeted temperature.

13. The method of claim 1, wherein the administering the energy dose is performed using a photo-thermal energy apparatus.

14. A method of performing an energy-based dermatological procedure without use of an injectable anesthetic, the method comprising:
applying a topical anesthetic to an area of skin;
cooling the area of skin such that a surface temperature of the area of skin is maintained between 0 degrees Celsius and 15 degrees Celsius for a duration of at least 30 seconds; and
performing a dermatological procedure on the area of skin after the duration.

15. The method of claim 14, wherein the surface temperature of the skin is measured in real-time.

16. The method of claim 14, wherein cooling the area of skin comprises at least one selected from a group consisting of passing a stream of cold air over the area of skin, contacting the area of skin with a cold medium, and applying at least one application of an evaporative medium to the area of skin.

17. The method of claim 14, wherein the energy-based dermatological procedure includes at least one of skin resurfacing, acne treatment, electro-needling, hair removal, tattoo removal, tattooing, and varicose vein removal.

18. The method of claim 14, wherein applying the topical anesthetic to the area of skin comprises applying a first application of topical anesthetic and applying at least a second application of topical anesthetic.

19. The method of claim 14, wherein applying the topical anesthetic comprises allowing the topical anesthetic to dwell on the area of skin for at least 30 minutes.

20. The method of claim 14, wherein the skin is between 0 degrees and 15 degrees Celsius for at least 30 seconds.

21. The method of claim 14, wherein an active ingredient of the topical anesthetic comprises at least one selected from a group consisting of lidocaine, epinephrine, and prilocaine.
